(19) United States
(12) Patent Application Publication
(10) Pub. No.: US 2016/0161507 A1
(43) Pub. Date: Jun. 9, 2016

(54) Method and Prognostic Kit for Monitoring Multiple Sclerosis (MS)

(71) Applicant: DIANTI MS LIMITED, London (GB)

(72) Inventors: Gilles GUILLEMIN, Mount Colah (AU); Chai Kiat LIM, Mascot (AU); Bruce James BREW, Vaucluse (AU)

(21) Appl. No.: 14/905,647

(22) PCT Filed: Jul. 18, 2013
(86) PCT No.: PCT/IB2013/055902

§ 371 Date: Jan. 15, 2016

(51) Int. Cl. G01N 33/68

(52) U.S. Cl. CPC G01N 33/6896; G01N 2800/285; G01N 2333/70539; G01N 2800/52

(57) ABSTRACT

A method and prognostic kit for assessing severity of MS in a subject suffering from MS, or for monitoring progression of MS in a subject suffering from MS, or for monitoring the effect of therapy administered to a subject suffering from MS. In both the method and prognostic kit, the level of one or more kynurenine pathway compounds in a tissue or body fluid of the subject suffering from MS are compared with a reference value for the one or more kynurenine pathway compounds.

### FIELD OF THE INVENTION

The invention relates to a method for assessing the severity of multiple sclerosis (MS) in a subject suffering from MS. The invention also relates to a method for monitoring the progression of MS in a subject suffering from MS, and to a method for monitoring the effect of therapy administered to a subject suffering from MS.

In preferred embodiments, the invention relates to a prognostic kit for assessing the severity of multiple sclerosis (MS) in a subject suffering from MS. The prognostic kit can also monitor the progression of MS in a subject suffering from MS as well as monitoring the effect of therapy administered to a subject suffering from MS.

### BACKGROUND

Multiple Sclerosis (MS) is a chronic neurological disease that affects almost 2.5 million people worldwide. MS varies in clinical subtype and severity, and has been generally classified into three categories or subtypes: relapsing-remitting MS (RRMS); secondary progressive MS (SPMS); and primary progressive MS (PPMS). Relapsing remitting MS represents the early stages of the disease and is characterized by exacerbations that result in varying degrees of disability, followed by periods of remission. Approximately 60-80% of patients with RRMS gradually progress to SPMS where the period of remission become shorter and gradually absent. PPMS, the most severe form among the MS subtypes, present similar clinical manifestation as SPMS but do not proceed from RRMS. Patients with PPMS experience persistent exacerbation of the disease from onset of the disease without any period of remission.

Assessing the severity of MS, including the subtype of MS, is important because the selection of treatment methods for MS is often dependent on the stage and type of disease. In addition, being able to assess the severity of MS permits the attending clinician to monitor a treatment regime to establish whether a particular treatment is effective in treating the disease.

However, prior art methods for determining the severity of MS or monitoring the progression of MS require the use of Magnetic Resonance Imaging of the brain in combination with tests such as neurological tests. Magnetic Resonance Imaging equipment is expensive and can be inconvenient for patients who may, in some cases, be severely disabled.

What is needed is a convenient and reliable method for assessing the severity, and monitoring the progression, of MS in subjects suffering from MS.

### SUMMARY OF THE INVENTION

In a first aspect, the invention provides a method for assessing severity of MS in a subject suffering from MS, or for monitoring progression of MS in a subject suffering from MS, or for monitoring the effect of therapy administered to a subject suffering from MS, comprising comparing the level of one or more kynurenine pathway compounds in the tissue or body fluid, for example, in the serum or cerebrospinal fluid (CSF), of the subject suffering from MS with a reference value for the one or more kynurenine pathway compounds.

Typically, a sample of the tissue or body fluid is obtained from the subject and the level of the one or more kynurenine pathway compounds in the sample compared with the reference value. Typically, the sample is a body fluid sample. Typically, the body fluid sample is a serum sample.

In a second aspect, the invention provides a method for assessing severity of MS in a subject suffering from MS, or for monitoring progression of MS in a subject suffering from MS, or for monitoring the effect of therapy administered to a subject suffering from MS, comprising comparing the level of one or more kynurenine pathway compounds in a sample obtained from the subject suffering from MS, with a reference value for the one or more kynurenine pathway compounds.

In a third aspect, the invention provides a method for assessing severity of MS in a subject suffering from MS, comprising comparing the level of one or more kynurenine pathway compounds in a sample obtained from the subject suffering from MS with a reference value for the one or more kynurenine pathway compounds.

In a fourth aspect, the invention provides a method for monitoring progression of MS in a subject suffering from MS, comprising comparing the level of one or more kynurenine pathway compounds in a sample obtained from the subject suffering from MS with a reference value for the one or more kynurenine pathway compounds.

In a fifth aspect, the invention provides a method for monitoring the effect of therapy administered to a subject suffering from MS, comprising comparing the level of one or more kynurenine pathway compounds in a sample obtained from the subject suffering from MS with a reference value for the one or more kynurenine pathway compounds.

### BRIEF DESCRIPTION OF THE FIGURES

FIG. 1A is a graph showing the concentration of tryptophan in serum samples from subjects not suffering from MS (control), or suffering from RRMA, SPMA or PPMS (as indicated).

FIG. 1B is a graph showing the ratio of kynurenine concentration to tryptophan concentration in serum samples from subjects not suffering from MS (control), or suffering from RRMA, SPMA or PPMS (as indicated).

FIG. 1C is a graph showing the concentration of tryptophan, kynurenine and K/T ratio in serum samples from subjects not suffering from MS (control), or suffering from RRMS, SPMS-Active (relapsing) or SPMS-Not Active (remitting) (as indicated).

FIG. 1D is a graph showing the concentration of tryptophan, kynurenine and K/T ratio in CSF samples from subjects not suffering from MS (control), or suffering from RRMS, SPMS-Active (relapsing) or SPMS-Not Active (remitting) (as indicated).

FIG. 2A is a graph showing the concentration of quinolinic acid in serum samples from subjects not suffering from MS (control), or suffering from RRMA, SPMA or PPMS (as indicated).

FIG. 2B is a bar graph of the concentration of quinolinic acid in serum samples from subjects not suffering from MS (control), or suffering from RRMS, SPMS-Active (relapsing) or SPMS-Not Active (remitting) (as indicated).

FIG. 2C is a bar graph of the concentration of quinolinic acid in CSF samples from subjects not suffering from MS (control), or suffering from RRMS, SPMS-Active (relapsing) or SPMS-Not Active (remitting) (as indicated).

FIG. 3 shows immunohistochemical staining for (A) myelin (Laxal Fast Blue stain) (B) activated microglia (HLA-DR) and (C)-(D) Neurotoxin, QUIN and its isotype control, respectively.

FIG. 4 shows immunohistochemical staining showing QUIN expression in chronic plaque (A), acute plaque (B), control (C), and in normal tissue at basal level (D).

FIG. 5A is a graph showing the concentration of 3-hydroxykynurenine in serum samples from subjects not suffering from MS (control), or suffering from RRMS, SPMS or PPMS (as indicated).

FIG. 5B is a bar graph of the concentration of 3-hydroxykynurenine in serum samples from subjects not suffering from MS (control), or suffering from RRMS, SPMS-Active (relapsing) or SPMS-Not Active (remitting) (as indicated).

FIG. 6 is a graph of the concentration of various KP metabolites ratios showing the changes between disease subtypes.

### DETAILED DESCRIPTION OF THE INVENTION

The invention relates in one aspect to a method for assessing the severity of MS in a subject suffering from MS. The method can be used to assess the subtype of MS, e.g. progressive MS (SPMS or PPMS) compared to RRMS, or PPMS compared to SPMS, suffered by the subject, as well as the severity of MS of a particular subtype. The severity of the MS is assessed by comparing the level of one or more kynurenine pathway compounds in a subject suffering from MS to a reference value for the one or more kynurenine pathway compounds.

The invention relates in another aspect to a prognostic kit for assessing the severity of MS in a subject suffering from MS. The prognostic kit can be used to assess the subtype of MS, e.g. progressive MS (SPMS or PPMS) compared to RRMS, or PPMS compared to SPMS, suffered by the subject, as well as the severity of MS of a particular subtype. The severity of the MS is assessed by comparing the level of one or more kynurenine pathway compounds in a subject suffering from MS to a reference value for the one or more kynurenine pathway compounds.

As used herein, the expression “kynurenine pathway compound” refers to a compound that is a substrate, product or metabolite of the kynurenine pathway. Kynurenine pathway compounds include tryptophan, kynurenine, kynurenic acid, 3-hydroxykynurenine, 3-hydroxy-anthranilic acid, picolinic acid, and quinolinic acid. In one form, the kynurenine pathway compound may be a kynurenine pathway metabolite. The kynurenine pathway metabolite may be a neurotoxic kynurenine pathway metabolite or a neuroprotective kynurenine pathway metabolite. An example of a neurotoxic kynurenine pathway metabolite is quinolinic acid.

Examples of neuroprotective kynurenine pathway metabolites include kynurenic acid and picolinic acid. In one embodiment, the kynurenine pathway compound is quinolinic acid. In another embodiment, the kynurenine pathway compound is picolinic acid. In a further embodiment, the kynurenine pathway compound is kynurenic acid.

As used herein, the term “subject” refers to a human. Humans are the only species known to suffer from MS.

The inventors have found a correlation between levels of kynurenine pathway compounds in cerebrospinal fluid (CSF) and serum and the severity of MS in subjects suffering from MS. In this regard, the inventors have found that the level of kynurenine pathway compounds such as quinolinic acid, 3-hydroxykynurenine, kynurenic acid and picolinic acid vary significantly during the progression of MS, and the variation in these compounds is correlated with the severity of MS.

Thus, by determining the level of these kynurenine pathway compounds in the CSF or serum of a subject suffering from MS, it is possible to assess the severity of the MS in the subject at any particular time, to monitor the progression of MS, or to monitor the effect of therapy administered to the subject.

Prior to the present invention, studies of quinolinic acid production in rats in which experimental allergic encephalomyelitis (EAE) was induced showed that quinolinic acid levels increased in the CNS of rats suffering from EAE (Flanagan et al. (1995) Journal of Endocrinology, 64:1192-1196). However, Flanagan et al. (1995) did not detect differences in levels of quinolinic acid in serum samples from rats suffering from EAE compared with rats not suffering from EAE.

Moreover, the EAE model in rats does not exhibit the same progression as MS in humans. Thus, it is not possible to correlate the level of compounds in the EAE model with disease severity and progression of MS in humans.

As described herein, the inventors have found that the kynurenine pathway compound quinolinic acid is elevated in serum and CSF of subjects suffering from MS as compared to subjects not suffering from MS, and that the quinolinic acid levels increase with increasing severity of the disease. Thus, the level of quinolinic acid in tissue or body fluid of a subject suffering from MS, can be used as a marker to indicate the severity of MS suffered by the subject at the time the level of quinolinic acid in the tissue or body fluid is determined.

The inventors have further found that the kynurenine pathway compounds kynurenic acid and picolinic acid are elevated in serum and CSF of subjects suffering from relapsing-remitting MS (RRMS) as compared to subjects not suffering from MS, and that the level of kynurenic acid and picolinic acid decreases with increasing severity of the disease. Thus, the level of kynurenic acid and/or picolinic acid in tissue or body fluid of a subject suffering from MS, can be used as a marker to indicate the severity of MS suffered by the subject at the time the level of kynurenic acid and/or picolinic acid in the tissue or body fluid is determined.

The inventors have further found that the kynurenine pathway compound 3-hydroxykynurenine is elevated in serum and CSF of subjects suffering from relapsing-remitting MS (RRMS) as compared to subjects not suffering from MS, and that the level of 3-hydroxykynurenine increases with increasing severity of the disease. Thus, the level of 3-hydroxykynurenine in tissue or body fluid of a subject suffering from MS, can be used as a marker to indicate the severity of MS suffered by the subject at the time the level of 3-hydroxykynurenine in the tissue or body fluid is determined.

Elevated levels of quinolinic acid and 3-hydroxykynurenine and lower levels of kynurenic acid and picolinic acid indicate that a subject may be suffering from MS. However, as many other neurodegenerative diseases also present with increased quinolinic acid and 3-hydroxykynurenine levels, and decreased kynurenic acid and picolinic acid levels, a sample from a subject showing elevated quinolinic acid and 3-hydroxykynurenine levels, and decreased kynurenic acid and picolinic acid levels, is not in itself determinative of a diagnosis of MS.

In one embodiment, the one or more kynurenine pathway compounds is a single kynurenine pathway compound, typically selected from the group consisting of quinolinic acid, picolinic acid, kynurenic acid and 3-hydroxykynurenine. More typically, the one or more kynurenine pathway compounds is quinolinic acid.

In another embodiment, the one or more kynurenine pathway compounds is a combination of kynurenine pathway compounds selected from the group consisting of quinolinic acid, picolinic acid, kynurenic acid, 3-hydroxykynurenine and tryptophan.

The level of one or more kynurenine pathway compounds in the tissue or body fluid of a subject suffering from MS may be assessed or monitored by obtaining a sample of the tissue or body fluid from the subject suffering from MS. Typically, the sample is a body fluid sample. The body fluid sample may be, for example, a CSF sample or a serum sample. Typically, the sample is a serum sample. In this regard, the inventors have found that the level of kynurenine pathway compounds in a serum sample from a subject suffering from MS can be used to assess the severity of the MS in the subject, to monitor the progression of MS, or to monitor the effect of therapy administered to a subject suffering from MS.

Thus, the kynurenine pathway compound can be used as a serum marker of severity of MS. The ability to use a serum sample provides a relatively convenient and rapid means by which to assess or monitor MS in a subject. As mentioned above, prior to the present invention, no convenient methods were available for assessing the severity of MS or monitoring the progression of MS.

The inventors have also found that the levels of the kynurenine pathway compounds vary in subjects of different ethnicities. For example, there is a clear difference in the gross levels of kynurenine pathway compounds between subjects of Asian descent compared with Caucasian and African ethnicity. However, the ratio of change in the levels of the kynurenine pathway compounds is consistent within each distinct ethnicity and allows for prognostic analysis and assessment of severity and progression in a subject suffering from MS.

Methods for obtaining samples such as CSF and serum samples from subjects are known in the art.

Once a sample has been obtained from the subject suffering from MS, the level of one or more kynurenine pathway compounds in the sample is compared with a reference value.

The term “level” refers to an indication of abundance.

Thus, “the level of one or more kynurenine pathway compounds” refers to an indication of the abundance of one or more kynurenine pathway compounds. The level of one or more kynurenine pathway compounds may be a measure of the amount of the one or more kynurenine pathway compounds per unit weight or volume. The level of one or more kynurenine pathway compounds may be a ratio, such as a ratio of the amount of the one kynurenine pathway compounds relative to the amount of another kynurenine pathway compound or some of the component in the tissue or body fluid.

In one embodiment, the level of the one or more kynurenine pathway compounds is the concentration of the one or more kynurenine pathway compounds. The concentration of quinolinic acid may be measured in any manner that is suitable for measuring concentrations of quinolinic acid in tissue or body fluids, for example, in CSF or serum samples.

Examples of suitable methods include mass-spectrometry and gas chromatography such as those described in Smythe et al., Concurrent quantification of quinolinic, picolinic, and nicotinic acids using electron-capture negative-ion gas chromatography-mass spectrometry, Anal. Biochem. 301 (1) (Feb. 1 2002), pp. 21-26, fluorometric analysis such as those described in Journal of Health Science (2009) 55(2): 242-248.

The concentration of picolinic acid may be measured in any manner that is suitable for measuring concentrations of picolinic acid in tissue or body fluids, for example, in CSF or serum samples. Examples of suitable methods include mass-spectrometry and gas chromatography such as those described in Smythe et al. Anal. Biochem. 301 (1) (Feb. 1, 2002), pp. 21-26.

The concentration of kynurenic acid may be measured in any manner that is suitable for measuring concentrations of kynurenic acid in tissue or body fluids, for example, in CSF and serum samples. Examples of suitable methods include HPLC, such as those described in The Journal of Neuroscience, Nov. 21, 2007, 27(47):12884-12892.

The concentration of tryptophan may be measured in any manner that is suitable for measuring concentrations of tryptophan in tissue or body fluids, for example, in CSF or serum samples. Examples of suitable methods include HPLC, such as those methods described in The Journal of Neuroscience, Nov. 21, 2007, 27(47):12884-12892.

The concentration of 3-hydroxykynurenine is measured using method adapted from The Journal of Chromatography B, 1996, 675:157-161. See page 22 for details. This method however is only limited to serum samples only.

The level of the one or more kynurenine pathway compounds in the tissue or body fluid of the subject suffering from MS is compared with a reference value for one or more kynurenine pathway compounds.

In one embodiment, the reference value for the one or more kynurenine pathway compounds is a value that represents the level of the one or more kynurenine pathway compounds in a tissue or body fluid, typically the same tissue or body fluid, from a subject not suffering from MS or suffering from MS of predetermined severity.

The reference value may be a predetermined standard value or may be a reference value obtained specifically for the comparison. The reference value may be the level of the one or more kynurenine pathway compounds in a reference sample from a subject not suffering from MS or suffering from MS of predetermined severity. As used herein, a “subject suffering from MS of predetermined severity” is a subject suffering from MS in which the severity of the MS is known.

The reference sample may be from a subject not suffering from MS. By comparing the level of the one or more kynurenine pathway compounds in a sample obtained from a subject suffering from MS with the level of the one or more kynurenine pathway compounds from a reference sample obtained from a subject not suffering from MS, it is possible to assess the severity of the disease, or to monitor the progression of the disease relative to a subject without disease.

The reference sample may be from a subject suffering from MS of a predetermined severity. By comparing the level of the one or more kynurenine pathway compounds in a sample obtained from a subject suffering from MS with the level of the one or more kynurenine pathway compounds from a reference sample obtained from a subject suffering from MS of a predetermined severity, it is possible to assess the severity of the disease, or to monitor the progression of the disease, relative to a subject with MS of known severity.

In other embodiments, the reference value represents the level of the one or more kynurenine pathway compounds in the tissue or body fluid of the subject suffering from MS at an earlier time. In such embodiments, the reference value is typically the level of the one or more kynurenine pathway compounds in a reference sample obtained from the subject suffering from MS at an earlier time. By comparing the level of the one or more kynurenine pathway compounds in a sample obtained from a subject suffering from MS with the level of the one or more kynurenine pathway compounds from a reference sample obtained from the same subject at an earlier time, it is possible to monitor whether the disease has progressed to a more severe form.

The severity of MS may be classified as relapsing-remitting MS (RRMS) or progressive MS (PMS). Progressive MS may be further classified as secondary progressive MS (SPMS) or primary progressive MS (PPMS). It will be understood by those skilled in the art that relapsing remitting MS is a less severe form of MS than secondary progressive, which is in turn a less severe form of MS than primary progressive MS.

In various embodiments:

(a) the reference value represents the level of picolinic acid or kynurenic acid in the tissue or body fluid of a subject not suffering from MS, and the MS is classified as relapsing-remitting MS when the level of picolinic acid or kynurenic acid in the tissue or body fluid of the subject suffering from MS is elevated relative to the reference value;
(b) a first reference value represents the level of quinolinic acid in the tissue or body fluid of a subject not suffering from MS and a second reference value represents the level of quinolinic acid in the tissue or body fluid from a patient suffering from secondary progressive MS, and the MS is classified as relapsing-remitting MS when the level of quinolinic acid in the tissue or body fluid of the subject suffering from MS is elevated relative to the first reference value and reduced relative to the second reference value;
(c) the reference value represents the level of picolinic acid or kynurenic acid in the tissue or body fluid of a subject suffering from relapsing-remitting MS, and the MS is classified as progressive when the level of picolinic acid or kynurenic acid in the tissue or body fluid of the subject suffering from MS is reduced relative to the reference value;
(d) the reference value represents the level of quinolinic acid in the tissue or body fluid of a subject suffering from relapsing-remitting MS, and the MS is classified as progressive when the level of quinolinic acid in the tissue or body fluid of the subject suffering from MS is elevated relative to the reference value;
(e) the reference value represents the level of picolinic acid or kynurenic acid in the tissue or body fluid of a subject suffering from secondary progressive MS, or a subject not suffering from MS, and the MS is classified as primary progressive MS (PPMS) when the level of picolinic acid or kynurenic acid in the tissue or body fluid of the subject suffering from MS is reduced relative to the reference value;
(f) the reference value represents the level of quinolinic acid in the tissue or body fluid of a subject suffering from secondary progressive MS, and the MS is classified as primary progressive MS (PPMS) when the level of quinolinic acid in the tissue or body fluid of the subject suffering from MS is elevated relative to the reference value;
(g) a first reference value represents the level of tryptophan in the tissue or body fluid of a subject not suffering from MS, and a second reference value represents the level of picolinic acid and/or kynurenic acid in the tissue or body fluid of a subject not suffering from MS, and the MS is classified as secondary progressive MS when the level of tryptophan in the tissue or body fluid of the subject suffering from MS is reduced relative to the first reference value and the level of picolinic acid and/or kynurenic acid in the tissue or body fluid of the subject suffering from MS is reduced relative to the second reference value.
(h) the reference value represents the level of 3-hydroxykynurenine in the tissue or body fluid of a subject not suffering from MS, and the MS is classified as relapsing-remitting MS, secondary progressive MS or primary progressive MS when the level of 3-hydroxykynurenine in the tissue or body fluid of the subject suffering from MS is elevated relative to the reference value;
(i) a first reference value represents the level of 3-hydroxykynurenine in the tissue or body fluid of a subject not suffering from MS, a second reference value represents the level of 3-hydroxykynurenine in the tissue or body fluid from a patient suffering from remitting phase secondary progressive MS (SPMS-NA) or relapse-remitting MS (RRMS), and a third reference value represents the level of 3-hydroxykynurenine in the tissue or body fluid of a subject suffering from relapse phase secondary progressive MS (SPMS-A), and the MS is classified as remitting phase secondary progressive MS (SPMS-NA) or relapse-remitting MS (RRMS) when the level of 3-hydroxykynurenine in the tissue or body fluid of the subject suffering from MS is elevated relative to the first reference value and reduced relative to the second reference value.

Typically, in embodiments (a) to (i) above, the level of the one or more kynurenine pathway compounds in the tissue or body fluid of the subject suffering from MS is the concentration of the one or more kynurenine pathway compounds in the tissue or body fluid of the subject suffering from MS.

Typically, in embodiments (a) to (i) above, the reference value represents the concentration of the one or more kynurenine pathway compounds in the tissue or body fluid of the subject not suffering from MS, or suffering from SPMS or RRMS.

More typically, in embodiments (a) to (i) above, the level of the one or more kynurenine pathway compounds in the tissue or body fluid of the subject suffering from MS is the concentration of the one or more kynurenine pathway compounds in the tissue or body fluid of the subject suffering from MS, and the reference value is the concentration of the one or more kynurenine pathway compounds in the tissue or body fluid of the subject not suffering from MS, or suffering from SPMS or RRMS.

It is envisaged that the method described above may be used to monitor the progression of MS. In this regard, the level of kynurenine pathway compounds in tissue or body fluids of the subject suffering from MS can be determined at various time intervals and the severity of the disease assessed at each time interval using the method described above in order to establish whether the severity of the disease is increasing.

In one form, the progression of MS can be monitored by comparing the level of the one or more kynurenine pathway compounds in tissue or body fluid of a subject suffering from MS to the level of the one or more kynurenine pathway compounds in tissue or body fluid of the subject at an earlier time. In this way, progression of the MS can be monitored based on whether the level of the one or more kynurenine pathway metabolites in the tissue or body fluid of the subject is elevated or reduced relative to the previously determined levels in the tissue or body fluid of the subject.

In one form, the method comprises assessing the severity of MS in a subject suffering from MS, or monitoring the progression of MS in a subject suffering from MS, or monitoring the effect of therapy administered to a subject suffering from MS, as described above, and selecting a therapy for the treatment of MS based on the outcome of that assessment or monitoring.

Also envisaged is a method of monitoring the effect of therapy on a subject suffering from MS. In this regard, the method described herein may be used to monitor the severity of the MS following therapy to determine whether the severity of the disease decreases or if the rate of increase in the severity of the disease is reduced, following treatment with the therapy.

### Examples

### Materials and Methods

### Patients

### Serum and CSF Samples for Analytical Studies

Samples used in this study were obtained from two sources in US: (1) Accelerated cure project for MS (ACPMS) and (2) Human brain and spinal fluid resource center (HBSFRC, UCLA). MS serum samples provided by ACPMS were from a repository of 733 MS subjects with various subtypes including, relapsing-remitting MS (RRMS), secondary progressive MS (SPMS) and primary progressive MS (PPMS) together with 50 control serum from healthy subjects. Diagnosis of MS had been assessed and evaluated based on expanded disability status scale (EDSS) and MRI scans that were provided by the ACPMS repository. The samples were screened for any medication known to affect the kynurenine pathway or had received steroid therapy over the past six months from the date of sample collection had been excluded in this study. 88 MS samples were used in this study based on the selected criteria (see Table 1 for more details).

TABLE 1
ACPMS subject population

| | | | Age | EDSS |
|---|---|---|---|---|
| Group | No. | Female:Male | (years + SD) | (+SD) |
| Control | 50 | 36:14 | 45.22 + 11.62 | Not applicable |
| RRMS (Total) | 51 | 36:19 | 43.43 + 9.47 | 2.71 + 1.7 |
| EDSS 0-3 | 38 | 25:13 | 42.47 + 10.04 | 1.88 + 0.71 |
| EDSS 3.5-5.5 | 6 | 4:2 | 49 + 7.62 | 3.67 + 0.26 |
| EDSS >6 | 7 | 3:4 | 43.86 + 6.34 | 6.36 + 0.24 |
| SPMS (Total) | 20 | 15:5 | 53.45 + 9.73 | 5.175 + 2.01 |
| EDSS 0-3 | 5 | 4:1 | 61.2 + 8.41 | 2.4 + 0.82 |
| EDSS 3.5-5.5 | 4 | 4:0 | 53.5 + 1.92 | 4.5 + 0.82 |
| EDSS >6 | 11 | 7:4 | 49.91 + 10.33 | 6.68 + 0.81 |
| PPMS (Total) | 17 | 13:4 | 52.24 + 8.79 | 4.74 + 2.14 |
| EDSS 0-3 | 5 | 3:2 | 50.4 + 11.39 | 2.1 + 0.42 |
| EDSS 3.5-5.5 | 4 | 4:0 | 51.75 + 5.38 | 4.25 + 0.96 |
| EDSS >6 | 8 | 6:2 | 53.63 + 9.25 | 6.63 + 0.92 |

TABLE 2
HBSFRC subject population

| | | | Age |
|---|---|---|---|
| Group | No. | Female:Male | (years + SD) |
| Control | 10 | 7:3 | 49.4 + 9.35 |
| RRMS - Remission | 10 | 7:3 | 49.2 + 15.99 |
| SPMS - Inactive | 10 | 7:3 | 49.7 + 9.04 |
| SPMS - Active | 10 | 7:3 | 50.9 + 8.48 |

Serum samples from MS patients with matching CSF were obtained from HBSFRC. Further verification between differences for MS stages (early stages of MS versus progressive MS) and active status (active versus inactive) of the disease had been considered. 10 cases of each MS subtypes consisting of RRMS in remitting, SPMS-inactive, SPMS-active and healthy subjects were used in this study for further illustration (see Table 2 for more details).

### Neuropathology of MS Post-Mortem Brain Tissue

The brain from a 49-year-old male with suspected MS and a control brain from a 48-year old male with no significant neuropathology were used in the study. Both brains were suspended in 20% formaldehyde for 4 weeks and then sectioned in the coronal plane. Sections were taken from the following regions; frontal, temporal and occipital corticles, the cerebellum, and brain stem.

### Chemicals

All chemicals were obtained from Sigma-Aldrich (Castle Hill, New South Wales, Australia) unless or otherwise stated. Acids, bases and acetonitrile used in the application for quantification of the KP metabolites were analytical grade and were obtained from commercial supplier (Ajax fine Chem).

### HPLC Quantification of KP Metabolites

### Sample and Standards Preparation

Working standards used for calibration curves were prepared from stock solutions (1 mM of respective KP metabolites) dissolving in ultra-pure water (Barnstead Easypure II, Thermo Scientific, New South Wales, Australia). The stock solutions were prepared on a weekly basis while the working standards were prepared freshly on daily basis. Serum samples were deproteinized by addition of an equal volume of 10% trichloroacetic acid, mixed and then centrifuged at 12,000 rpm for 5 min at 4° C. The supernatant were then collected and used for analysis. Prior to quantification, all standards and samples were filtered through a syringe filter (4 mm, 0.45 μm PTFE, Waters Corporation, New South Wales, Australia).

### Tryptophan and Kynurenine Detection

TRP and KYN were quantified concurrently using Agilent 1200 series HPLC system (Agilent Technologies, New South Wales, Australia) complete with fluorescence and multi-wavelength detector in accordance to a method described previously Smythe et al. Anal. Biochem. 301 (1) (Feb. 1, 2002), pp. 21-26. Briefly, the standards and samples were applied to an Agilent Zorbax Eclipse XDB-C18 (5 μm, 250×4.6 mm i.d.) column (Biolab, Victoria, Australia) at an injection volume of 30 μl. The mobile phase consists of 0.1M ammonia acetate, at pH 4.65 is filtered through a filtering system (0.2 μm nylon membrane, Milipore, New South Wales, Australia) prior to usage and pumped isocratically at a flow rate of 1 ml/min. TRP was measured using a fluorescence detection at an excitation wavelength of 254 nm and an emission of 404 nm while KYN was detected using a multi-wavelength UV detection at 365 nm.

### Kynurenic Acid Detection

KYNA was assayed by Agilent 1200 series HPLC system (Agilent Technologies, New South Wales, Australia) equipped with fluorescence detector as outlined in Smythe et al. Anal. Biochem. 301 (1) (Feb. 1, 2002), pp. 21-26 with minor changes. Briefly, 30 μl of the standards and samples were applied to an Agilent Zorbax Eclipse XDB-C18 (5 μm, 150×4.6 mm i.d.) column. KYNA was eluted isocratically at a flow rate of 0.8 ml/min with a mobile phase consisting of 50 mM sodium acetate with 0.25M of zinc acetate and 2.25% (v/v) acetonitrile. Mobile phase is prepared freshly and filtered prior to use. KYNA is detected using fluorescence detector at an excitation wavelength of 344 nm and an emission wavelength of 388 nm.

The intra- and inter-assay coefficient of variations ranged from 5% to 7% for all the metabolites detected using the HPLC.

### GCMS Quantification of KP Metabolites

### Picolinic Acid and Quinolinic Acid Detection

PIC and QUIN were simultaneously measured using Gas chromatography-mass spectrometry (GC/MS), which was previously described by Smythe G A et al, 2002. Standards or samples (50 μl) were added to glass tubes (100×10 mm, Biolab, Victoria, Australia) together with equal volume of internal standards (d3-quinolinic acid and d4-picolinic acid). The mixtures were allowed to dry (Savant SpeedVac) leaving residues which are then mixed with trifluoroacetic anhydride and hexafluoroisopropanol (1:1; 120 μl). The tubes were sealed with a Telfon-lined cap (Biolab, Victoria, Australia) and allow to derivatize to produce hexafluoroisopropyl ester of the respective acids (i.e. PIC and QUIN) for 45 min at 60° C. The derivatized products dissolved in toluene (final volume of 250 μl), were then washed insolubly in 5% sodium bicarbonate (1 ml) and water (1 ml), dried and filter through sailane treated glass wool (Grace davison discovery sciences, Victoria, Australia) packed with anhydrous sodium sulfate (approx. 50 mg each samples) and transferred into autosampler vials prior to injection (1 μl) into the GC/MS (Agilent Technologies). The spectrometer is operated in electron capture negative ionization mode with ion selectivity of 273, 277, 467, 470, for PIC-derivative, d4-PIC, QUIN-derivative and d3-QUIN, respectively. Finally, concentrations of PIC and QUIN in samples were determined from the calibration curves based on the peak area ratio of the derivatives to their respective internal standards within the samples. The limits of detection were less than 1 fmol at signal-to-noise ratio of greater than 10:1.

3-hydroxykynurenine Detection

3HK was assayed by Agilent 1200 series HPLC system (Agilent Technologies, New South Wales, Australia) equipped with UV detector as outlined in Hervéet al. J. Chromatography B. 1996, 675, pp. 157-161 with minor changes. Briefly, 50 μl of the standards and samples were applied to an Agilent Zorbax Eclipse XDB-C18 (3.5 μm, 150×4.6 mm i.d.) column. 3HK was eluted isocratically at a flow rate of 0.5 ml/min with a mobile phase consisting of 0.1M sodium acetate at pH 4.65. Mobile phase is prepared freshly and filtered prior to use. 3HK is detected using multi-wavelength UV detector at 365 nm.

The intra- and inter-assay coefficient of variations ranged from 5% to 7% for 3HK within a detection limit of 10 nM or greater using the HPLC.

### Immunohistochemistry

### Brain Section Preparation

The following antibodies were used; HLA-DR mAb (1:100 dilution, DAKO), QUIN mAb (IgG1, 1:100 dilution, Chemicon Millipore). Paraffin sections 5 μm in thickness were acquired and floated from water bath (HD Scientific) at 38° C. onto Superfrost Ultra Plus (Thermo Scientific) glass slides. Sections were dried in a tissue-drying oven (Medite) at 45° C. overnight. Sections were then hydrated by transfer through two changes of xylene then two changes of absolute alcohol, through graded alcohol concentrations (90% and 70% respectively) and then to water. Endogenous peroxidases were blocked by placing the sections in a 3% hydrogen peroxide (H₂O₂)/methanol solution for 20 min at RT.

### HLA-DR Staining

Sections for HLA-DR antibody staining were placed in citric acid buffer pH 6.0 and antigenic retrieval was induced at 120° C. for 20 min in an autoclave (Siltex). Sections were then washed in 0.1 M tris (hydroxymethyl) aminomethane (TRIS) buffered saline pH 7.6 with sterile horse serum (Invitrogen) at a final concentration of 3% for 5 min at RT. Sections were circled with a PAP pen (DAKO Cytomation, Copenhagen, Denmark). Antibody was then applied to the sections and incubated for 1 hr at RT. Antibody was washed off sections and then placed in TRIS buffer pH 6.0 for a further 5 min at RT. The Envision (DAKO) link polymer was then applied to sections and incubated for 30 min at RT. Peroxidase labelling was visualized by incubating sections in 0.03% H₂O₂/0.05% 3,3-diaminobenzidine tetrachloride (DAB, Sigma D5637) in 0.1M TRIS buffer pH 7.6 for 2 min at RT followed by water rinse. Sections were finally counterstained in Harris's Haematoxylin for 2 min then differentiated for 3 sec in 1% acid alcohol and blued in Scott's Blueing solution. Sections were then dehydrated, cleared in xylene and then mounted in Pertex mounting medium (HD Scientific).

### QUIN Staining

Sections for QUIN antibody staining were placed briefly in 10% sterile horse serum in 0.1M Tris-HCl buffer pH 7.5, 0.15M NaCl (TNB) and 0.5% blocking reagent (Perkin Elmer, Zaventem, Belgium). Sections were then washed in 0.1M Tris-HCl buffer pH 7.5, 0.3M Nacl, 0.05% Tween-20 (TNT) 3 times for 3 min each wash. Sections were circled with a PAP pen and two drops of avidin solution was added to each slide for 15 min then washed in TNT twice three min each wash. Two drops of biotin was added to each section and incubated for 15 min. Slides were washed in TNT twice for 3 min each wash then autoclaved at 120° C. for 20 min in citric acid buffer pH 6.0. Slides were left to cool in the retrieval solution following autoclaving and then washed 3 times each for 3 min in TNT. Sections placed in 10% horse serum in TNB for 30 min. QUINN antibody was applied to test sections at 1:100 dilution while mouse IgG1 (1:15 dilution in TNB) was applied to isotype control sections which was the equivalent concentration of protein to the antibody used. Horse serum (10% in TNB) was tipped off the sections and antibody or mouse IgG1 was applied to appropriate sections for 1 hr. Sections were then washed in TNT for 3 min 3 times. Secondary antibody (biotinylated anti-mouse) was then applied (1:200 dilution) to all sections for 30 at RT. Sections were washed 3 times for 3 min and Avidin-Biotin complex (ABC elite, Vector Laboratories) was applied for 30 min and washed again for 3 min 3 times. Biotinyl Tramide (1:50 dilution, Invitrogen) was applied to the sections for 10 min then washed off with TNT 3 times 3 min. Sections were then incubated for 30 min at RT in SA-HRP (1:100 dilution, Invitrogen) and then developed in DBA 3 min at RT. Sections were washed in running tap water for 20 min then counterstained in Harris's Haematoxylin for 30 sec. Sections were washed in water and dipped once in acid alcohol (1%) then rinsed in water and blued (Scott's Blueing solution) for 1 min washed in water and dehydrated to xylene then permanently mounted in Fastmount.

### H&E Staining

Sections were taken to tap water and stained in Harris's Hematoxylin for 5 min. Sections were then washed in water and differentiated for 3 sec in 1% acid alcohol. Sections were blued in Scott's Blueing solution, washed again in tap water, dehydrated in alcohol, cleared in xylene and mounted with Pertex.

### Luxol Fast Blue/Cresyl Violet Staining

Sections were taken to water then rinsed in 95% alcohol and stained in pre heated (60° C.) LFB working solution for 2 hr. Sections were then left to cool at RT for 1 hr. Sections were then place into Lithium Carbonate pH 10.5, 4° C. with agitation 10 min. Differentiate slides in 70% alcohol 75 sec. Wash in running tap water 10 min. Rinse in Distilled water and counterstain with 0.1% Cresyl Violet 10 min. Sections were quick wash in tap water, then dehydrated slowly through three changes of absolute alcohol to remove excess Cresyl Violet then cleared and mounted.

### Statistical Analysis

All data were expressed as median with standard deviation throughout the text. Statistical comparisons between groups with n>15 for significance of differences were performed using parametric 1-way analysis of variance (ANOVA) followed by post-hoc Turkey's comparison analysis. A p-value <0.05 was considered statistically significant. For n<15, non-parametric Kruskall-Wallis of variance was used with Mann-Whitney's comparison analysis. Due to comparison analysis, we use p-value <0.01 as a significant threshold while p-value <0.05 to exhibit a trend. Graphs charts were illustrated using GraphPad Prism 5 software package while statistical analyses were performed using SPSS version 17.0.

### Results

### KP Activation in MS Progression

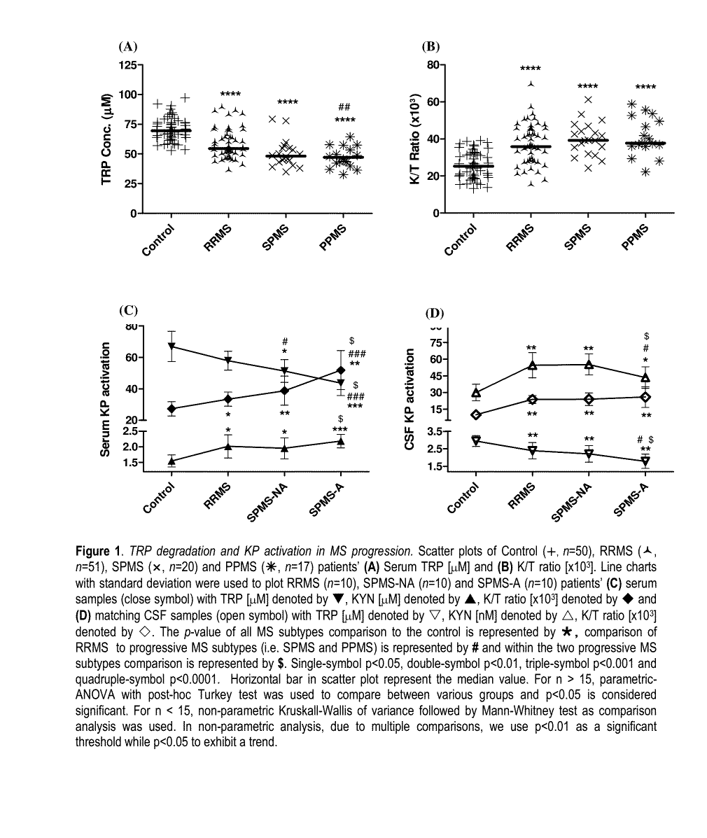

Three parameters had been used to assess the KP activation in both serum and CSF samples from MS patients, namely TRP, KYN and the K/T ratio. The inventor's data indicate that TRP, the first substrate that drives the KP was significantly decreased in serum of all MS subtypes compared to control (FIG. 1). The inventors did not see any differences in the correlation of TRP degradation to the severity of the disease within MS subtypes from serum samples and CSF samples. However, statistical analysis revealed that there is a trend exhibiting decreasing TRP from progressive MS compared to early stages of the disease (FIG. 1 A&C—p<0.05). The result was further validated in matching CSF samples (open triangle in FIG. 1 D) showing decrement in the TRP concentration. Also, we see a similar trend of decreasing TRP comparing the active form to its non-active form of SPMS in FIG. 1 C&D (p<0.05).

The immediate catabolite of the TRP, KYN was increased in MS as well. Samples obtained from HBSFRC shows that an increasing trend of KYN from serum samples (p<0.05) and a significant increase in the active form of MS. This increase of KYN was also significant in the matching CSF samples (p<0.01). However, we see a decrease in KYN from active SPMS in CSF as oppose to its matching serum samples.

There was no significant difference for serum KYN observed between the MS subtypes and the control except for RRMS (p<0.05, data not presented) in ACPMS samples.

The resulting K/T ratio depicting the inverse relation of KYN and TRP was increased in MS in comparison to the control. K/T ratio used to assess the activation of the KP and indicative of IDO activity suggests that the KP is indeed activated in MS. Furthermore, a similar trend was also observed in matching CSF samples being significantly higher than its control. However, our data shows no significant correlation in the elevation of K/T ratio to the severity of the disease.

### Neuroprotective KP Metabolites in MS Progression

The levels of neuroprotective KP metabolites obtained for serum and CSF is shown in Tables 3 and 4 below.

TABLE 3
Tryptophan, kynurenine and K/T ratio
in serum of MS patients from ACPMS.

| | TRP | KYN | K/T Ratio |
|---|---|---|---|
| | (μM) | (μM) | (×10³) |
| Control (Mean) | 67.85 + 11.04 | 1.77 + 0.38 | 26.89 + 8.01 |
| RRMS (Mean) | 57.89 + 12.72 | 2.06 + 0.51 | 36.94 + 10.99 |
| EDSS - 0-3 | 60.86 + 13.2 | 2.08 + 0.53 | 35.67 + 11.6 |
| EDSS - 3.3-5.5 | 46.01 + 3.1 | 1.82 + 0.45 | 39.31 + 8.72 |
| EDSS - >6 | 51.94 + 5.11 | 2.17 + 0.45 | 41.80 + 8.39 |
| SPMS (Mean) | 50.44 + 11.63 | 1.94 + 0.24 | 40.12 + 9.09 |
| EDSS - 0-3 | 63.29 + 14.09 | 1.95 + 0.2 | 31.73 + 6.42 |
| EDSS - 3.3-5.5 | 42.92 + 7.16 | 1.72 + 0.23 | 40.74 + 7.93 |
| EDSS - >6 | 47.34 + 6.8 | 2.03 + 0.22 | 43.70 + 8.49 |
| PPMS (Mean) | 47.09 + 8.75 | 1.91 + 0.5 | 41.15 + 10.27 |
| EDSS - 0-3 | 55.26 + 7.79 | 1.87 + 0.55 | 33.63 + 7.8 |
| EDSS - 3.3-5.5 | 41.35 + 6.21 | 1.72 + 0.34 | 42.33 + 11.01 |
| EDSS - >6 | 44.85 + 7.19 | 2.04 + 0.56 | 45.26 + 9.69 |

TABLE 4
Serum and matching CSF of Tryptophan, kynurenine and K/T ratio of MS patients from HBSFRC.

| | TRP | | KYN | | K/T Ratio | |
|---|---|---|---|---|---|---|
| | Serum | CSF | Serum | CSF | Serum | CSF |
| | (μM) | (μM) | (μM) | (nM) | (×10³) | (×10³) |
| Control | 66.95 + 9.61 | 3.04 + 0.23 | 1.55 + 0.2 | 30.05 + 7.51 | 27.27 + 4.58 | 9.95 + 2.7 |
| RRMS | 57.85 + 6.02 | 2.39 + 0.46 | 2.01 + 0.37 | 54.56 + 11.44 | 33.49 + 4.47 | 23.79 + 3.64 |
| SPMS Non | 51.34 + 7.24 | 2.21 + 0.47 | 1.95 + 0.34 | 55.28 + 9.45 | 38.78 + 9.27 | 23.99 + 5.73 |
| active | | | | | | |
| SPMS | 43.62 + 7.87 | 1.79 + 0.4 | 2.18 + 0.22 | 43.52 + 9.68 | 51.87 + 12.43 | 25.92 + 9.22 |
| Active | | | | | | |

### KYNA Concentration

The neuroprotective KP metabolites, KYNA was found to be elevated in serum of RRMS patients. However, with the progression of the disease, we observed a decrease in this neuroprotective metabolite in both SPMS and PPMS. Furthermore, a trend exhibiting the decrement in KYNA was more pronounce in the active form as compare to its counterpart in SPMS serum and matching CSF (p<0.05).

### PIC Concentration

PIC the other neuroprotective KP metabolite follows a similar trend to the KYNA. PIC was found to be increase in serum of RRMS patients but decreases in SPMS and PPMS patients. As well, the decrease in PIC was further extend to the active form of SPMS in comparison to its non-active form (p<0.01).

### Neurotoxin QUIN Production in MS Progression

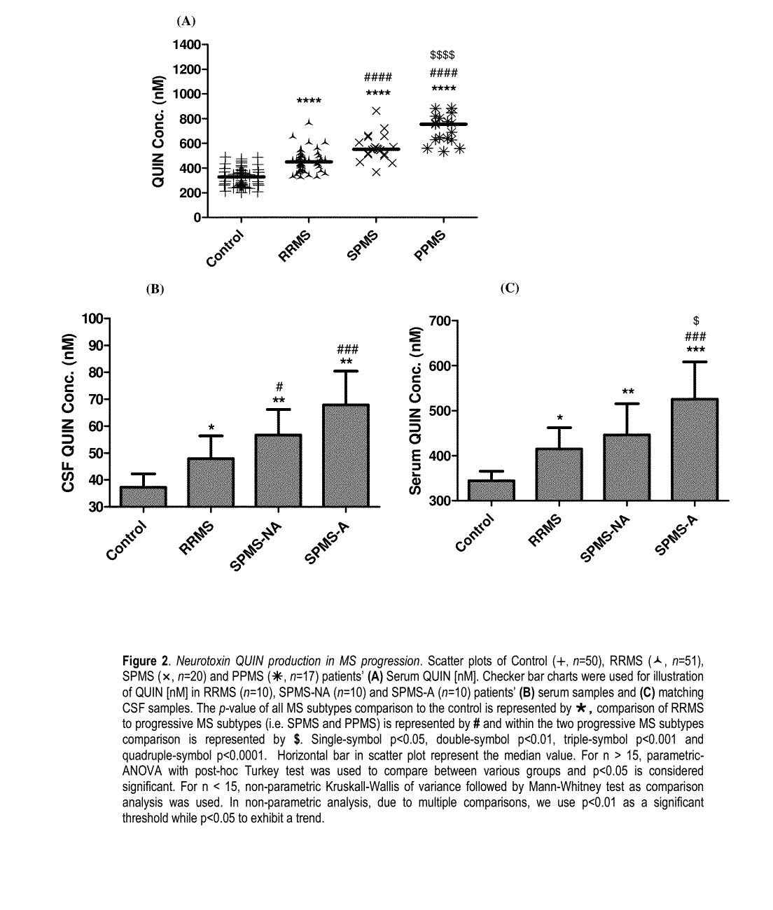

The level of quinolinic acid in serum and CSF from subjects of different MS severity is shown in Tables 5 and 6 below and FIG. 2.

TABLE 5
Neuroprotective KP metabolites in
serum of MS patients from ACPMS.

| | KYNA | PIC |
|---|---|---|
| | (nM) | (nM) |
| Control | 57.38 + 10.46 | 393.29 + 76.14 |
| RRMS (Mean) | 76.53 + 18.13 | 457.97 + 91.2 |
| EDSS - 0-3 | 82.87 + 15.12 | 431.04 + 81.22 |
| EDSS - 3.3-5.5 | 62.71 + 11.67 | 493.23 + 35.66 |
| EDSS - >6 | 53.97 + 13.3 | 573.89 + 77.81 |
| SPMS (Mean) | 42.65 + 8.23 | 388.10 + 66.94 |
| EDSS - 0-3 | 37.36 + 3.63 | 401.33 + 39.74 |
| EDSS - 3.3-5.5 | 50.77 + 9.44 | 396.51 + 43.92 |
| EDSS - >6 | 42.10 + 7.48 | 379.02 + 84.24 |
| PPMS (Mean) | 41.93 + 10.27 | 246.90 + 51.54 |
| EDSS - 0-3 | 53.12 + 6.17 | 241.94 + 53.82 |
| EDSS - 3.3-5.5 | 44.71 + 8.02 | 249.81 + 50.33 |
| EDSS - >6 | 33.54 + 4.15 | 248.53 + 57.48 |

TABLE 6
Serum and matching CSF of neuroprotective
KP metabolites in MS patients from HBSFRC

| | KYNA | | PIC | |
|---|---|---|---|---|
| | Serum | CSF | Serum | CSF |
| | (nM) | (nM) | (nM) | (nM) |
| Control | 58.74 + 6.98 | 2.28 + 0.48 | 414.29 + 30.83 | 53.23 + 11.96 |
| RRMS | 71.19 + 13.04 | 1.64 + 0.39 | 442.58 + 59.72 | 48.39 + 9.96 |
| SPMS | 59.94 + 12.57 | 1.69 + 0.51 | 392.27 + 41.94 | 56.34 + 7.27 |
| Non | | | | |
| active | | | | |
| SPMS | 44.7 + 9.29 | 1.2 + 0.34 | 325.16 + 40.74 | 36.39 + 8.78 |
| Active | | | | |

QUIN concentration was elevated in all MS subtypes in comparison to the control. The elevation of QUIN production in serum also correlates to the disease severity (p<0.0001). This increment of QUIN was also found in matching CSF samples. In addition, there seem to be a trend in significant increase of QUIN in progressive MS compared to the less severe form of MS (i.e. RRMS).

The inventor's found that 3HK was significantly increased in serum of all MS subtypes when compared to healthy controls. Further, the inventors found an increasing trend of 3HK in progressive MS in comparison to RRMS but did not reach a statistical significant (p=0.045). Interestingly, in another cohort of MS samples, the inventors found that 3HK was significantly increased in serum of relapse phase SPMS (SPMS-A) samples when compare to its remitting phase (i.e. SPMS-NA and RRMS). This suggests that the activated KP may be prone towards production of 3HK during relapse of the disease and likely leading to downstream production of the other neurotoxin QUIN.

### Immunohistochemical Study

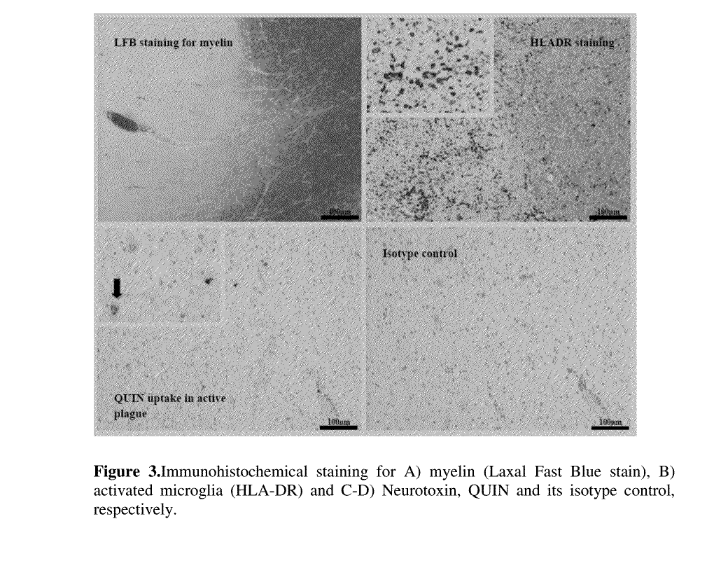

Microscopic sections showed extensive and multiple demyelinating plaques throughout the cerebrum, brain stem and cerebellum. Periventricular plaques within the frontal, temporal and occipital cortex showed complete demyelination with perivascular lymphocytic cuffing present and some reactive gliosis. In some areas the plaques appeared to be of a longer age showing no residual perivascular lymphocytes. There was no other significant cortical pathology. Basal ganglia and diencephalon showed focal areas of perivascular demyelinating plaques. Laxal Fast Blue with crystal violet staining of the basal ganglion showed both chronic and acute plagues observed in the MS case. The staining (see FIG. 3) illustrates extensive demyelination on the left side as compared to the normal myelination on the right side was observed. HLA-DR showed prolific infiltration of activated microglia into demyelinated area of the acute plaques. Whereas in chronic plaques, there was absence of extensive microglia infiltration as shown in FIG. 3B.

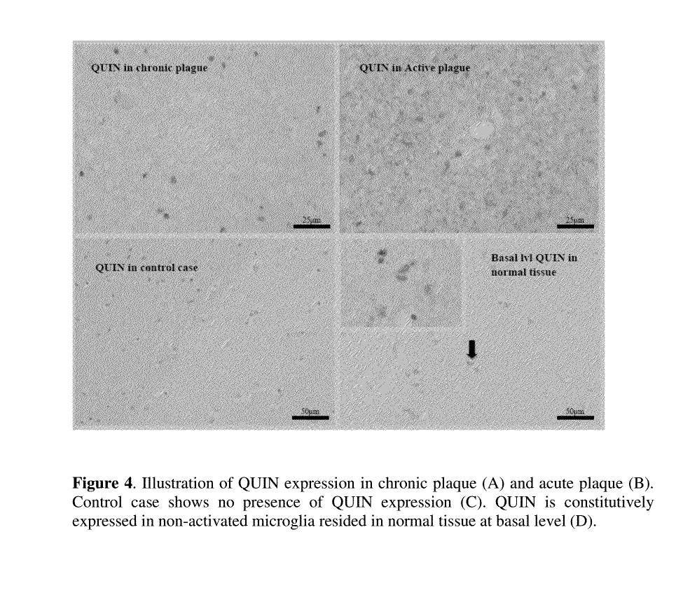

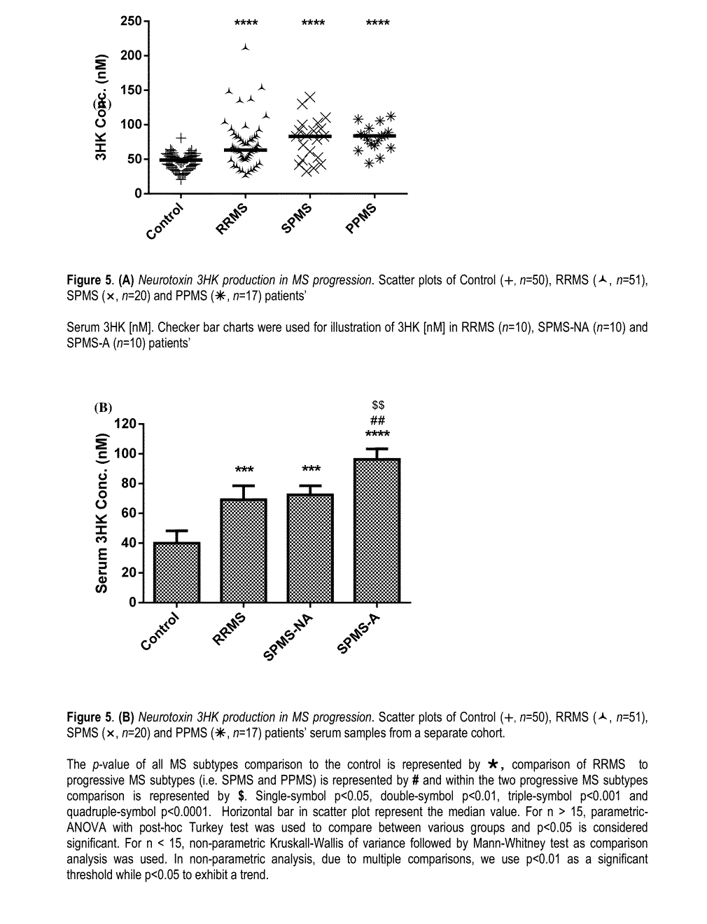

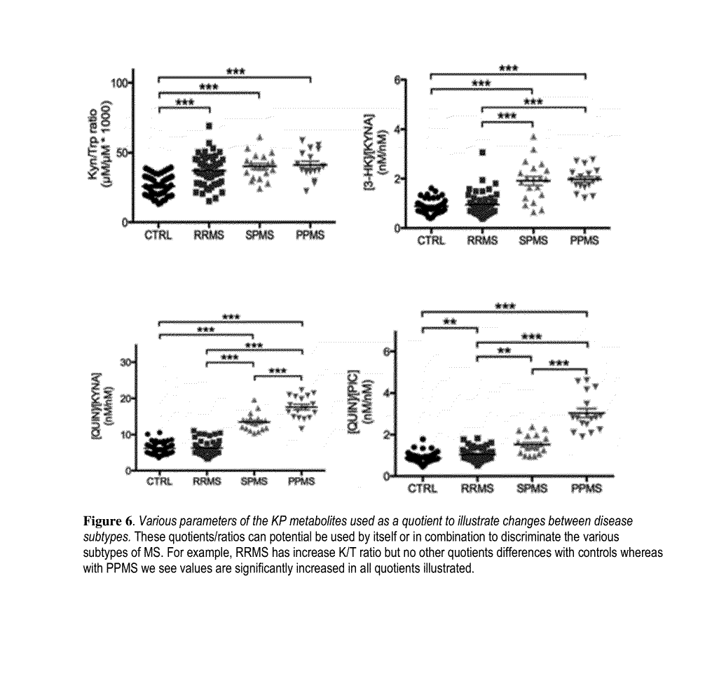

QUIN was also present in the MS brain section. In acute plaque as define by presence of perivascular lymphocytic cuffing, cytoplasmic expression of QUIN was seen in neuronal cells (see FIG. 3C). In addition, QUIN expression was found in stippled pattern in the brain parenchyma in regions of the brain showing acute plaque (FIG. 4A) but no staining of QUIN was found in chronic plaques (FIG. 4B). QUIN isotype control (FIG. 3D) in acute plaques did not show any neuronal expression of QUIN. Furthermore, when compared to a control brain section (FIG. 4C), QUIN expression was not observed in both grey and white matter. However, QUIN was exclusively and constitutively expressed at basal levels in resting microglia of normal brain tissue (FIG. 4D).

TABLE 7
levels of the kynurenine pathway compounds in a tissue
or body fluid of the subject suffering from MS.

| | K/T Ratio | 3HK/KYNA | QUIN/KYNA | QUIN/PIC |
|---|---|---|---|---|
| | (×10³) | Ratio | Ratio | Ratio |
| Control | 25.77 ± 1.05 | 0.88 ± 0.04 | 6.18 ± 0.23 | 0.86 ± 0.03 |
| RRMS | 36.94 ± 1.54 | 0.95 ± 0.07 | 6.24 ± 0.28 | 1.03 ± 0.04 |
| SPMS | 40.12 ± 2.03 | 1.91 ± 0.18 | 13.48 ± 0.5 | 1.52 ± 0.1 |
| PPMS | 41.15 ± 2.49 | 1.97 ± 0.11 | 17.61 ± 0.74 | 3.04 ± 0.21 |

### DISCUSSION

The data shows that the neuroprotective KP metabolites, namely KYNA and PIC, were increased in the early stages of MS, and significantly decreased in progressive MS.

QUIN is elevated in all MS subtypes. This increase correlates with the progression of MS, especially in the CNS and serum. In early stages of the disease, only a moderate increase of QUIN as compared to control is observed. In progressive MS, the data indicates that QUIN was markedly elevated.

3-hydroxykynurenine is elevated in all MS subtypes compared with healthy controls. In particular, 3-hydroxykynurenine levels are significantly increased during the relapse phase of MS, which is evident in the elevated levels of 3-hydroxykynurenine during the relapse phase (SPMS-active) when compared with the levels of 3-hydroxykynurenine in the remitting phase (SPMS-non-active) of MS.

Staining of QUIN in MS brain section revealed that in initial stages of acute plaque formation, there is release of QUIN into the parenchyma. To justify that the QUIN expression in the parenchyma is not due to background staining, an isotype control for QUIN staining was set up and demonstrated that QUIN staining is absent in the isotype control. In addition, it was observed that QUIN was not expressed in either white or gray matter of control case with no significant neuropathology.

1. A method for assessing severity of MS in a subject suffering from MS, or for monitoring progression of MS in a subject suffering from MS, or for monitoring the effect of therapy administered to a subject suffering from MS, comprising comparing the level of one or more kynurenine pathway compounds in a tissue or body fluid of the subject suffering from MS with a reference value for the one or more kynurenine pathway compounds.

2. A method of claim 1, wherein the reference value represents the level of the one or more kynurenine pathway compounds in the tissue or body fluid of a subject not suffering from MS.

3. A method of claim 1, wherein the reference value represents the level of the one or more kynurenine pathway compounds in the tissue or body fluid of a subject suffering from MS of a predetermined severity.

4. A method of claim 1, wherein the reference value represents the level of the one or more kynurenine pathway compounds in the tissue or body fluid of the subject suffering from MS at an earlier time.

5. A method of any one of claims 1 to 4, wherein the one or more kynurenine pathway compounds are selected from the group consisting of tryptophan, kynurenic acid, 3-hydroxykynurenine, picolinic acid and quinolinic acid.

6. A method of claim 5, wherein the one or more kynurenine pathway compounds is quinolinic acid and the severity of the MS is classified as relapsing-remitting MS (RRMS) or progressive MS, wherein progressive MS is further classified as secondary progressive MS (SPMS), relapse phase secondary progressive MS (SPMS-A), remitting phase secondary progressive MS (SPMS-NA) or primary progressive MS (PPMS).

7. A method of claim 5, wherein one or more kynurenine pathway compounds is picolinic acid and/or kynurenic acid and the severity of the MS is classified as relapsing-remitting MS (RRMS) or progressive MS, wherein progressive MS is further classified as secondary progressive MS (SPMS), relapse phase secondary progressive MS (SPMS-A), remitting phase secondary progressive MS (SPMS-NA) or primary progressive MS (PPMS).

8. A method of claim 5, wherein the one or more kynurenine pathway compounds is 3-hydroxykynurenine and the severity of the MS is classified as relapsing-remitting MS (RRMS) or progressive MS, wherein progressive MS is further classified as secondary progressive MS (SPMS), relapse phase secondary progressive MS (SPMS-A), remitting phase secondary progressive MS (SPMS-NA) or primary progressive MS (PPMS).

9. A method of claim 6, wherein a first reference value represents the level of quinolinic acid in the tissue or body fluid of a subject not suffering from MS and a second reference value represents the level of quinolinic acid in the tissue or body fluid from a patient suffering from secondary progressive (SPMS), and wherein the MS is classified as relapsing-remitting MS (RRMS) when the level of quinolinic acid in the tissue or body fluid of the subject suffering from MS is elevated relative to the first reference value, and reduced relative to the second reference value.

10. A method of claim 7, wherein the reference value represents the level of picolinic acid or kynurenic acid in the tissue or body fluid of a subject not suffering from MS, and wherein the MS is classified as relapsing-remitting MS (RRMS) when the level of picolinic acid or kynurenic acid in the tissue or body fluid of the subject suffering from MS is elevated relative to the reference value.

11. A method of claim 8, wherein the reference value represents the level of 3-hydroxykynurenine in the tissue or body fluid of a subject not suffering from MS, and the MS is classified as relapsing-remitting MS (RRMS), secondary progressive MS (SPMS) or primary progressive MS (PPMS) when the level of 3-hydroxykynurenine in the tissue or body fluid of the subject suffering from MS is elevated relative to the reference value.

12. A method of claim 6, wherein the reference value represents the level of quinolinic acid in the tissue or body fluid of a subject suffering from relapsing-remitting MS (RRMS), and wherein the MS is classified as progressive when the level of quinolinic acid in the tissue or body fluid of the subject suffering from MS is elevated relative to the reference value.

13. A method of claim 7, wherein the reference value represents the level of picolinic acid or kynurenic acid in the tissue or body fluid of a subject suffering from relapsing-remitting MS (RRMS), and wherein the MS is classified as progressive MS when the level of picolinic acid or kynurenic acid in the tissue or body fluid of the subject suffering from MS is reduced relative to the reference value.

14. A method of claim 8, wherein a first reference value represents the level of 3-hydroxykynurenine in the tissue or body fluid of a subject not suffering from MS, a second reference value represents the level of 3-hydroxykynurenine in the tissue or body fluid from a patient suffering from remitting phase secondary progressive MS (SPMS-NA) or relapse-remitting MS (RRMS), and a third reference value represents the level of 3-hydroxykynurenine in the tissue or body fluid of a subject suffering from relapse phase secondary progressive MS (SPMS-A), wherein the MS is classified as remitting phase secondary progressive MS (SPMS-NA) or relapse-remitting MS (RRMS) when the level of 3-hydroxykynurenine in the tissue or body fluid of the subject suffering from MS is elevated relative to the first reference value and reduced relative to the second reference value.

15. A method of claim 5, wherein the levels of the one or more kynurenine pathway compounds in a tissue or body fluid of the subject suffering from MS are in the ratios set out in the table below:—

| | K/T Ratio | 3HK/KYNA | QUIN/KYNA | QUIN/PIC |
|---|---|---|---|---|
| | (×10³) | Ratio | Ratio | Ratio |
| RRMS | 36.94 ± 1.54 | 0.95 ± 0.07 | 6.24 ± 0.28 | 1.03 ± 0.04 |
| SPMS | 40.12 ± 2.03 | 1.91 ± 0.18 | 13.48 ± 0.5 | 1.52 ± 0.1 |
| PPMS | 41.15 ± 2.49 | 1.97 ± 0.11 | 17.61 ± 0.74 | 3.04 ± 0.21 |
| | | | | [EL2] |

16. A method of claim 6, wherein the reference value represents the level of quinolinic acid in the tissue or body fluid of a subject suffering from SPMS, and wherein the MS is classified as PPMS when the level of quinolinic acid in the tissue or body fluid of the subject suffering from MS is elevated relative to the reference value.

17. A method of claim 7, wherein the reference value represents the level of picolinic acid or kynurenic acid in the tissue or body fluid of a subject suffering from SPMS, or a subject not suffering from MS, and wherein the MS is classified as PPMS when the level of picolinic acid or kynurenic acid in the tissue or body fluid of the subject suffering from MS is reduced relative to the reference value.

18. A method of claim 5, wherein the reference value represents the level of tryptophan and the level of picolinic acid or kynurenic acid in the tissue or body fluid of a subject not suffering from MS, and wherein the MS is classified as SPMS when the level of tryptophan and the level of picolinic acid or kynurenic acid in the tissue or body fluid of the subject suffering from MS is reduced relative to the reference value.

19. A method of claim 1, wherein the level of the one or more kynurenine pathway compounds in the tissue or body fluid is determined by taking a sample of the tissue or body fluid of the subject suffering from MS and determining the level of the one or more kynurenine pathway compounds in the sample.

20. A method of claim 19, wherein the sample is a CSF or a serum body fluid sample.

21. A prognostic kit for assessing severity of MS in a subject suffering from MS, or for monitoring progression of MS in a subject suffering from MS, or for monitoring the effect of therapy administered to a subject suffering from MS, comprising comparing the level of one or more kynurenine pathway compounds in a tissue or body fluid of the subject suffering from MS with a reference value for the one or more kynurenine pathway compounds.

22. A prognostic kit of claim 21 comprising:
(a) reagents for determining the level of one or more kynurenine pathway compounds in a biological sample of a patient; and
(b) information to correlate the level of the one or more kynurenine pathway compounds to one or more reference values to assess the severity of MS in a subject suffering from MS.

23. A prognostic kit of claim 22 including an enzyme-linked immuno sorbent assay (ELISA) test utilising TRP, KYN, KYNA, 3HK, PIC and QUIN monoclonal antibodies.
